(19) United States
(12) Patent Application Publication
(10) Pub. No.: US 2010/0149486 A1
(43) Pub. Date: Jun. 17, 2010

(54) DEVICE FOR MEASURING THE ANGLE BETWEEN FAR SIGHT AND NEAR SIGHT ON A PATIENT WEARING SPECTACLES

(75) Inventor: Jean-Philippe Sayag, Paris (FR)

Correspondence Address: YOUNG & THOMPSON, 209 Madison Street, Suite 500, Alexandria, VA, 22314, US

(73) Assignee: ACEP FRANCE, ISSY LES MOULINEAUX (FR)

(21) Appl. No.: 12/161,022

(22) PCT Filed: Jan. 31, 2007
(86) PCT No.: PCT/FR2007/050721

§ 371 Date: Sep. 22, 2008

(30) Foreign Application Priority Data
Jan. 31, 2006 (FR) 06/00876

(51) Int. Cl. A61B 3/10

(52) U.S. Cl. 351/204

(57) ABSTRACT

A device for measuring the angle Δ formed between a first position, corresponding to far sight, of a patient wearing a pair of spectacles, and a second position, corresponding to near sight, includes: a recording unit (22) for recording the orientation of the pair of spectacles in each of the two positions, and a processing member (30) for determining the angle Δ as a function of the orientations, the device being characterized in that it includes a visual reference element (26) for defining the attitude of the patient in far sight.

The present invention relates to a device for measuring the angle Δ formed between a first position corresponding to far sight, of a patient wearing a pair of spectacles, and second position corresponding to near sight.

In the field of the spectacle trade, different methods and devices intended for determining the physiological parameters of a patient are known, with a view to a spectacle frame. With some of the devices it is possible to provide automatic recording of the measurements by means of a motion-picture camera and to determine the resulting appropriate measurements with means for processing the obtained images. With the conducted measurements, an optician may then make correction lenses depending on the patient.

The invention relates to correction lenses allowing far sight and near sight. A pair of spectacles including such correction lenses is illustrated in FIG. 1. Each correction lens 2 comprises a first area 4 which occupies the upper portion and the side portions of the correction lens, and a second area 6 which occupies the central lower portion of the correction lens. The first area is the one used by the patient for far sight, whereas the second area is used for near sight. Both of these areas are generally separated by an intermediate area, a so-called progression channel, not shown in the figure. By near sight, is meant a sight at less than 80 cm, and in particular of the order of 40 cm, i.e., the sight used for reading a document held by the patient between his/her hands.

The distance d between the upper limit of the correction lens and the upper limit of the second area 6 varies depending on the patients. In order to determine it, the optician asks the patient to look away and then to look closely and he/she notes the difference in position of the spectacles of the patient between both sights, i.e., the amplitude of the vertical head motion of the patient between both sights. With the angular difference Δ between both of these positions, the optician may know the value of the appropriate distance d for the patient.

A drawback of the prior art is that the position of the spectacles in far sight is only determined in a very imperfect way. Indeed, the optician who asks the patient to stare in the distance does not exactly know what the patient is looking at. The latter might have his/her glance attracted by a particular object, the position of which does not exactly correspond to far sight. An uncertainty results from this on the appropriate position of the spectacles in far sight, this causes inaccuracy on the value of the distance d. This causes discomfort for the patient who is forced to adjust (move) the pair of spectacles when he/she passes from far sight to near sight or vice versa.

The object of the invention is to find a remedy to the drawbacks of the prior art and to propose a device with which the angle Δ may be measured in a more accurate way, and thus provide the patient with correction lenses for which the limit of the first and second areas is adapted to the sight of the latter.

Thus the object of the present invention is a device for measuring the angle Δ formed between a first position, corresponding to far sight, of a patient wearing a pair of spectacles, and a second position, corresponding to near sight, said device comprising a recording means capable of recording the orientation of the pair of spectacles in each of the two positions, and a processing means for determining the angle Δ as a function of said orientations, this device further including a visual reference element in order to define the attitude of the patient for the far sight measurement.

With the visual reference element the first position may be determined in an accurate way, and therefore the angle Δ between the positions corresponding to far and near sights.

Advantageously, the device comprises a positioning component which, during a measurement, is mechanically bound to the pair of spectacles, this positioning component including at least one reference mark, the recording means being able to record the orientation of said reference mark.

According to a preferred embodiment, the device includes a displacement means for adjusting the height of the recording means.

Advantageously, the recording means is a motion-picture camera and the processing means includes an image processing module, for example as a software package, in order to determine the orientation of the visual reference element in each of both positions and to infer the value of the angle Δ therefrom.

The visual reference element is preferably integral with the recording means.

As a non-limiting example, an embodiment of the present invention will be described hereafter, with reference to the appended drawing wherein:

FIG. 1, already described, illustrates a pair of spectacles, the correction lenses of which include an area for far sight and an area for near sight,

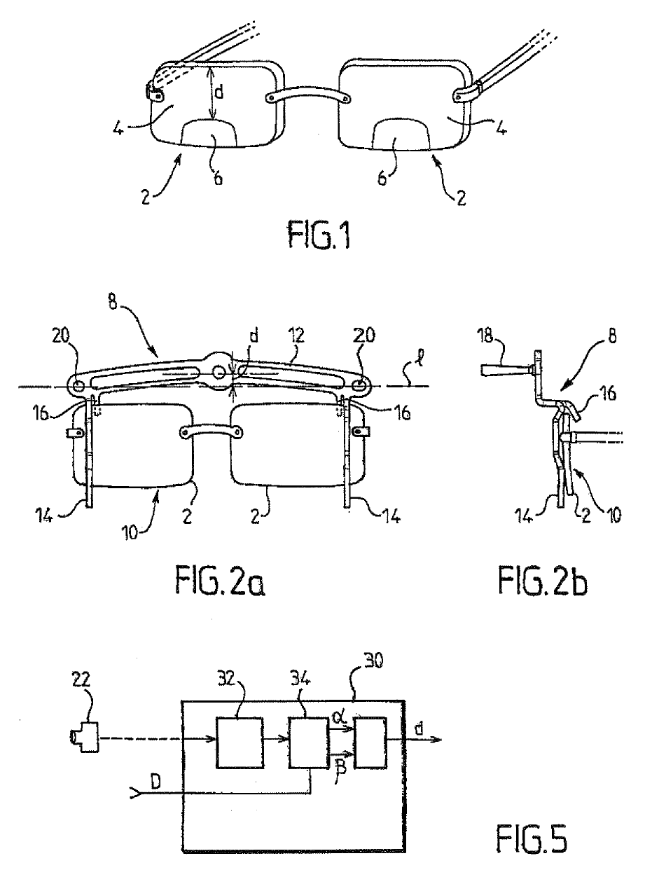

FIGS. 2a and 2b respectively illustrate in a front view and a profile view, a pair of spectacles provided with a positioning component,

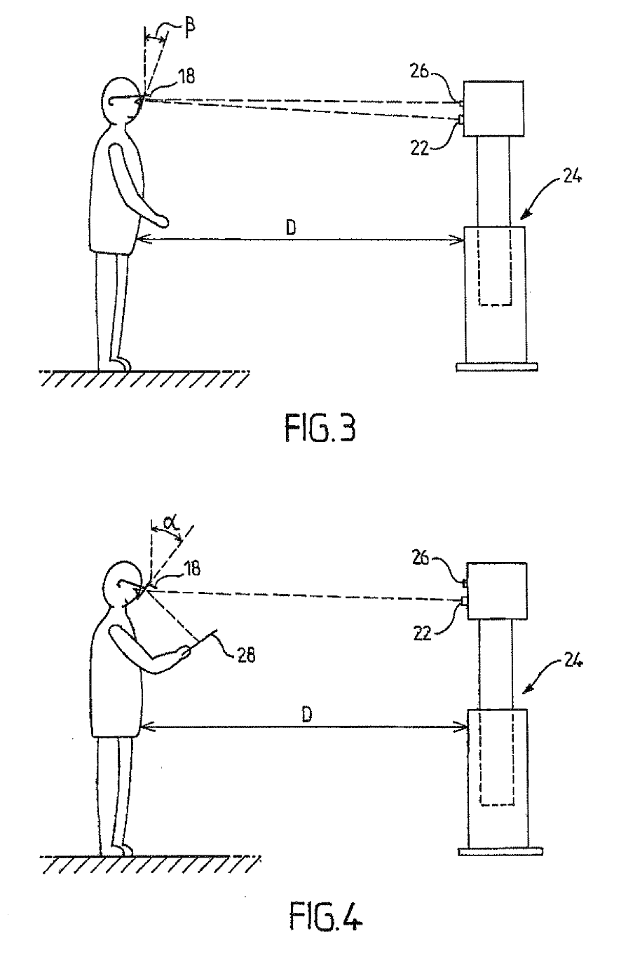

FIG. 3 illustrates the measurement of the orientation of the positioning component in far sight,

FIG. 4 illustrates the measurement of the orientation of the positioning component in near sight, and

FIG. 5 schematically illustrates the processing means of the device.

A positioning component 8 mounted on a pair of spectacles is illustrated in FIGS. 2a and 2b. This positioning component includes a substantially horizontal bridge 12, the ends of which are each provided with a first branch 14 against which the front face of a correction lens 2 will be supported and a second branch 16 which is supported on the rear face of the correction lens. The positioning component includes at least one reference mark 18. The orientation of the latter is recorded and determined by the measuring device. The difference in orientation—resulting from a different position of the head of the patient between the far sight and the near sight—enables the optician to accurately define for each patient the suitable value of the distance d (see FIG. 1) for this patient.

As this is illustrated in FIG. 2a, the positioning component may comprise additional reference marks such as reference marks 20 for checking the proper positioning of the pair of spectacles relatively to the horizontal. These reference marks 20 may also be used for measuring the orientation of the reference mark 18, the latter being a function of the height h of the reference mark 18 above the line 1 connecting the reference marks 20.

FIGS. 3 and 4 illustrate the use of the device according to the invention in order to determine the angle Δ and subsequently the distance d, in a known way.

This device comprises a positioning component mounted on the pair of spectacles as illustrated in FIGS. 2a and 2b. It comprises a recording means 22, for example a motion-picture camera or a still camera mounted on a column 24 forming a height-adjustable displacement means. In order to determine the orientation of the reference mark of the positioning component during far sight, the device includes a visual reference element 26 for the patient. This visual reference element is for example placed just above or just below the recording means. It may also be of the annular type encircling the lens of the recording means. It is not necessarily close to the recording means but may for example be attached to a wall which is found behind the recording means.

In the devices according to the prior art, the patient himself/herself selects a reference supposed to represent the position of his/her head in far sight. He/she may then take as a reference, a point which only corresponds imperfectly to what corresponds to his/her natural attitude in far sight.

With the invention, it is possible to get rid of this inaccuracy by forcing the glance of the patient onto the visual reference element.

The angle β formed by the mean plane of the correction lenses and the vertical is calculated by a processing means by analyzing in the captured image the orientation of the reference mark 18 (FIGS. 2a and 2b). This analysis may be performed automatically with an image processing software package.

The measuring method is again applied in the same way, for near sight, as illustrated in FIG. 4. The patient is prompted to look at a document 28, which he/she holds in the usual attitude which is his/her own for reading close up. The angle α formed between the mean plane of the correction lenses and the vertical is calculated by the processing means in the same way as the angle β. The value d (FIG. 1) for the correction lenses of the patient is then inferred from the angular difference Δ=α−β in a known way.

In FIGS. 3 and 4, reference was made to the angles formed by the mean plane of the correction lenses in far and near sight. It is clear that any other component of the pair of spectacles, such as the branches or any other component mechanically bound to the pair of spectacles, such as the reference mark 18, has different orientations between far sight and near sight, and that the difference of these orientations also forms an angular difference Δ.

It is therefore understood that measurement of the orientations in far sight and in near sight may be achieved by placing the patient sideways relatively to the recording means (the visual reference element for far sight as for it being always placed facing the patient), the processing means then determining the angle Δ by measuring the angular difference of a branch of spectacles or of the reference mark, or of any other portion of the pair of spectacles or of the positioning component, between far and near sights.

FIG. 5 schematically illustrates an embodiment of the processing means 30. It comprises a storage module 32 for storing in memory an image transmitted by the motion-picture camera 22 and an image processing module 34 for determining the orientation of the reference mark 18 in the stored image. This module may take into account the distance D separating the patient from the recording means. From the stored images corresponding to the attitude of the patient in far sight and in near sight, the processing means 30 successively determines the angles α and β, and infers the angular difference Δ=α−β therefrom and from this difference the value of the distance d.

1. A device for measuring the angle Δ formed between a first position, corresponding to far sight, of a patient wearing a pair of spectacles, and a second position, corresponding to near sight, said device comprising:
a recording means (22) capable of recording the orientation of the pair of spectacles in each of the two positions, and
a processing means (30) for determining the angle Δ as a function of said orientations,
said device being characterized in that it includes a visual reference element (26) for defining the attitude of the patient in far sight.

2. The device according to claim 1, characterized in that it comprises a positioning component (8) which, during a measurement, is mechanically bound to the pair of spectacles, this positioning component including at least one reference mark (18), the recording means (22) being capable of recording the orientation of said reference mark (18).

3. The device according to claim 1, characterized in that it comprises a displacement means (24) for adjusting the height of the recording means (22).

4. The device according to claim 1, characterized in that the recording means comprises a motion-picture camera (22).

5. The device according to claim 4, characterized in that the processing means (30) comprises an image processing module (34).

6. The device according to claim 1, characterized in that the visual reference element (26) is in the vicinity of the recording means (22).

7. The device according to claim 6, characterized in that the visual reference element (26) is above or below the recording means (22).

8. The device according to claim 2, characterized in that it comprises a displacement means (24) for adjusting the height of the recording means (22).

9. The device according to claim 2, characterized in that the recording means comprises a motion-picture camera (22).

10. The device according to claim 3, characterized in that the recording means comprises a motion-picture camera (22).

11. The device according to claim 2, characterized in that the visual reference element (26) is in the vicinity of the recording means (22).

12. The device according to claim 3, characterized in that the visual reference element (26) is in the vicinity of the recording means (22).

13. The device according to claim 4, characterized in that the visual reference element (26) is in the vicinity of the recording means (22).

14. The device according to claim 5, characterized in that the visual reference element (26) is in the vicinity of the recording means (22).
